(19) United States
(12) Patent Application Publication
(10) Pub. No.: US 2018/0035673 A1
(43) Pub. Date: Feb. 8, 2018

(54) A Mycoherbicide

(71) Applicant: The Secretary of State for Environment, Food and Rural Affairs, London (GB)

(72) Inventors: Marion Karin Seier, Ascot, Berkshire (GB); Harold Charles Evans, Camberley, Surrey (GB)

(21) Appl. No.: 15/555,105

(22) PCT Filed: Mar. 2, 2016
(86) PCT No.: PCT/GB2016/050553

§ 371 Date: Sep. 1, 2017

(30) Foreign Application Priority Data
Mar. 2, 2015 (GB) 1503510.8

(51) Int. Cl. A01N 63/04; C12N 1/14

(52) U.S. Cl. CPC A01N 63/04; C12N 1/14

(57) ABSTRACT

Use of a consortium of cells of the species Mycosphaerella polygoni-cuspidati or the like for the herbicidal control of a plant of the genus Fallopia. The cells in the consortium are all of the same mating type.

### FIELD OF THE INVENTION

The present invention relates to the use of a consortium of cells of the species Mycosphaerella polygoni-cuspidati or the like for the herbicidal control of a plant. The invention also relates to a method for herbicidal control of a plant of the genus Fallopia. In addition, the invention relates to a method of producing a consortium of cells of the species Mycosphaerella polygoni-cuspidati. The invention further relates to a composition for the herbicidal control of a plant of the genus Fallopia.

### BACKGROUND OF THE INVENTION

Introduced in the 19^{th} century for ornamental purposes, Japanese knotweed (Fallopia japonica) has spread dramatically throughout the UK and continental Europe, as well as many parts of the US and Canada. Today, the species is listed as one of the world's top 100 invaders.

In contrast to its native Japanese range, in its exotic ranges, Japanese knotweed grows aggressively, forming dense stands with severe impacts on native biodiversity. Rapid rhizomatous growth enables Japanese knotweed to force its way through concrete and tarmac with serious consequences to local infrastructures. The estimated annual cost to the British economy is currently £166 million (Williams et al. (2010) The Economic Cost of Invasive Non-Native Species on Great Britain; CABI E-UK report). Mechanical and/or chemical control of Japanese knotweed is both costly and labour-intensive. Thus, classical biological control of Japanese knotweed i.e. the introduction to its exotic ranges of co-evolved, host-specific natural enemies from its native range, which form self-sustaining populations, is an ideal additional tool for the integrated management of Japanese knotweed.

In addition, to Japanese knotweed (Fallopia japonica), the closely related species giant knotweed (Fallopia sachalinensis) and a hybrid between these two species, Fallopia×bohemica, also pose threats as invasive species.

The ascomycete fungus Mycosphaerella polygoni-cuspidati is a damaging, co-evolved natural enemy of Japanese knotweed which is found ubiquitously in Japan. Infection with the pathogen causes severe necrotic lesions on Japanese knotweed leaves leading to premature senescence and ultimately leaf drop. It was therefore believed that M. polygoni-cuspidati was a highly promising subject for classical biological control of Japanese knotweed. However, the problem with this proposal is that testing against a list of 91 native species revealed that M. polygoni-cuspidati causes non-target effects on certain other plant species. More specifically, M. polygoni-cuspidati causes non-target effects on the rare British native plant species Polygonum maritimum and also, to a lesser degree, the species Persicaria hydropiper. In particular, M. polygoni-cuspidati infection results in necrotic lesions and the formation of spermogonia on P. maritimum, which constitute the first stage of the ascomycete life cycle. Due to the potential risk posed to P. maritimum, the introduction of M. polygoni-cuspidati in the UK as part of a classical biological control programme against Japanese knotweed would be ill-advised in case there was inadvertent infection of, and thus risk to, P. maritimum or other native flora. It is also possible, and perhaps likely, that other countries in the exotic range of Japanese knotweed also have native species that would be at risk from M. polygoni-cuspidati. As a consequence, the use of M. polygoni-cuspidati as a classical control agent agent with a self-sustaining and augmenting field population was seen to be unviable.

The present invention arises out of the recognition that P. maritimum generally occupies a different habitat (i.e. a coastal habitat) compared with Japanese knotweed and that, therefore, if it were possible to limit the spread of M. polygoni-cuspidati then the fungus could be applied to Japanese knotweed as a mycoherbicide, without risking P. maritimum. The invention further arises from the recognition that M. polygoni-cuspidati is heterothallic (that is to say, mating between distinct isolates possessing complementary mating genes, MAT-1 and MAT-2, is required for the development of ascospores and for completion of its life cycle) and it has no asexual conidial stage. This unusual combination of characteristics has resulted in the surprising realization that if M. polygoni-cuspidati were applied in the form of only a single mating type then the fungus would not develop ascospores, and, as the fungus does not produce conidia either, this prevents the fungus from reproducing and spreading in the field beyond the site of application. The spread of M. polygoni-cuspidati can therefore be very closely controlled. Thus applying M. polygoni-cuspidati in the form of only a single mating type enables M. polygoni-cuspidati to be used as a herbicide against Japanese knotweed.

### SUMMARY OF THE INVENTION

According to a first aspect of the present invention, there is provided the use of a consortium of cells of the species Mycosphaerella polygoni-cuspidati or the like for the herbicidal control of a plant of the genus Fallopia, wherein the cells in the consortium are all of the same mating type.

Conveniently, the plant is of the species or hybrid: Fallopia japonica, Fallopia sachalinensis or Fallopia×bohemica preferably Fallopia japonica var. japonica.

Preferably, the cells are mycelium cells.

Advantageously, the Mycosphaerella polygoni-cuspidati cells in the consortium are all of mating type 1.

Alternatively, the Mycosphaerella polygoni-cuspidati cells in the consortium are all of mating type 2.

According to a second aspect of the present invention, there is provided a method for herbicidal control of a plant of the genus Fallopia comprising contacting the plant with a composition comprising a consortium of cells of the species Mycosphaerella polygoni-cuspidati or the like, wherein the cells in the consortium are all of the same mating type.

Conveniently, the method further comprises the step of maintaining the cells in an aqueous environment on the plant for at least 12 hours, preferably at least 24 hours.

Preferably, the method comprises contacting the leaves of the plant with the composition.

Advantageously, the plant is of the species or hybrid: Fallopia japonica, Fallopia sachalinensis or Fallopia×bohemica preferably Fallopia japonica var. japonica.

Coveniently, the cells are mycelium cells.

Preferably, the Mycosphaerella polygoni-cuspidati cells in the consortium are all of mating type 1.

Alternatively, the Mycosphaerella polygoni-cuspidati cells in the consortium are all of mating type 2.

According to a third aspect of the present invention, there is provided a method of producing a consortium of cells of the species Mycosphaerella polygoni-cuspidati wherein the cells in the consortium are all of the same mating type comprising the steps of:
i. isolating a first individual ascospore from a sample of ascospores of the species Mycosphaerella polygoni-cuspidati; and
ii. generating at least a first mycelium from said first isolated ascospore.

Conveniently, the method further comprises the step of:
iii. determining the mating type of said first mycelium.

Preferably, the method further comprises the steps of:
iv. isolating a second individual ascospore from a sample of ascospores of the species Mycosphaerella polygoni-cuspidati;
v. generating at least a second mycelium from said second isolated ascospore;
vi. determining the mating type of said second mycelium;
vii. confirming that the mating type of the first mycelium is the same as the mating type of the second mycelium;
viii. mixing the first and second mycelia.

Advantageously, step iii and/or step vi comprise detecting the presence of a biological marker specific for one mating type.

Conveniently, the biological marker is a polynucleotide of the sequence of SEQ. ID NO: 1 or 5 or a fragment thereof which is at least 20, preferably at least 50 nucleotides in length.

According to a fourth aspect of the present invention, there is provided a composition for the herbicidal control of a plant of the genus Fallopia comprising a consortium of cells of the species Mycosphaerella polygoni-cuspidati or the like, wherein the cells in the consortium are all of the same mating type.

Conveniently, the cells are mycelium cells.

Preferably, the Mycosphaerella polygoni-cuspidati cells in the consortium are all of mating type 1.

Alternatively, the Mycosphaerella polygoni-cuspidati cells in the consortium are all of mating type 2.

Preferably, the composition further comprises an adhesive agent and/or a wetting agent.

According to a fifth aspect of the present invention, there is provided a polynucleotide primer comprising the sequence of any one of SEQ. ID NOS: 9 to 12.

According to a sixth aspect of the present invention, there is provided a method for herbicidal control of a plant comprising contacting the plant with a composition comprising a consortium of cells of a fungal species which has the following characteristics:
i. the fungal species is capable of infecting the plant and causing necrosis in the plant;
ii. the fungal species has no asexual stage in its life cycle;
iii. the fungal species is heterothallic,
   wherein the cells of the fungal species in the consortium are all of the same mating type.

### Definitions

As used herein, the term “Mycosphaerella polygoni-cuspidati” or “M. polygoni-cuspidati” means a fungus in accordance with the taxonomy set forth in Kurose et al., (2009) Systematics of Mycosphaerella species associated with the invasive weed Fallopia japonica, including the potential biological control agent M. polygoni-cuspidati. Mycoscience 50, 179-189.

As used herein, the term “Mycosphaerella polygoni-cuspidati or the like” means the fungus Mycosphaerella polygoni-cuspidati and species having at least 98% similarity (or 98% sequence identity) in their ITS (internal transcribed spacer) region to the ITS of Mycosphaerella polygoni-cuspidati and meeting the following requirements: i) the fungus infects, and has a herbicidal effect on, a plant of the genus Fallopia; ii) the fungus cycles exclusively through its sexual stage (and has no asexual reproduction) and iii) the fungus is heterothallic.

As used herein, the term “herbicidal control” means reduction or elimination of plant health or growth and includes (but is not limited to) exfoliation and plant death.

As used herein, the term “a plant of the genus Fallopia” means any plant of the genus Fallopia. In some embodiments it is defined as any invasive plant of the genus Fallopia. It includes the species Fallopia japonica (which is also known as Japanese Knotweed) and Fallopia sachalinensis and the hybrid thereof, Fallopia×bohemica. It also includes other hybrid species of the aforementioned species, in particular other hybrids of Fallopia japonica.

As used herein, the term “consortium of cells” means a collection or mixture of cells. In some embodiments the term may be replaced with the term “aggregate of cells” throughout the specification.

As used herein, the term “mating type” means the characteristics that define two sexually compatible individuals.

The percentage “identity” between two sequences may be determined using the BLASTP algorithm version 2.2.2 (Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402) using default parameters. In particular, the BLAST algorithm can be accessed on the internet using the URL http://www.ncbi.nlm.nih.gov/blast/.

### FIGURES

FIG. 1 is a schematic diagram of the life cycle of M. polygoni-cuspidati.

FIG. 2 shows photographs of disease symptoms of M. polygoni-cuspidati (ex Mt Hiko, Japan) on Japanese knotweed at four points in the life cycle of M. polygoni-cuspidati.

FIG. 3 is a diagram of the life cycle of M. polygoni-cuspidati.

FIG. 4 is a photograph of disease symptoms of M. polygoni-cuspidati (ex Mt Hiko, Japan) on Japanese knotweed 2.5 months after inoculation with ascospores in quarantine greenhouse.

FIG. 5 is a photograph of disease symptoms of M. polygoni-cuspidati (ex Mt Hiko, Japan) on Japanese knotweed 2.5 months after inoculation with ascospores in quarantine greenhouse, top leaf with white label over it is the control leaf.

FIG. 6 is a photograph of Japanese knotweed leaf material infected with M. polygoni-cuspidati, bearing mature pseudothecia (right hand side) and leaf discs, conditioned in sterile distilled water for ascospore drop, affixed to petri dish lids for suspension over agar to obtain ascospore drop.

FIG. 7 is a photograph of PDA (potato dextrose agar) broth inoculated with mycelial plugs of M. polygoni-cuspidati after one week incubation on a shaker.

FIG. 8 is a photograph of macerated mycelium of M. polygoni-cuspidati used for inoculations.

FIG. 9 is a photograph of disease symptoms of M. polygoni-cuspidati on Japanese knotweed circa 3 weeks after inoculation with mycelium in quarantine greenhouse.

FIG. 10 is a photograph of disease symptoms of M. polygoni-cuspidati (ex Mt Hiko, Japan) on Japanese knotweed 1 month after inoculation with mycelial broth in quarantine greenhouse.

### DETAILED DESCRIPTION

Mycosphaerella polygoni-Cuspidati or the Like

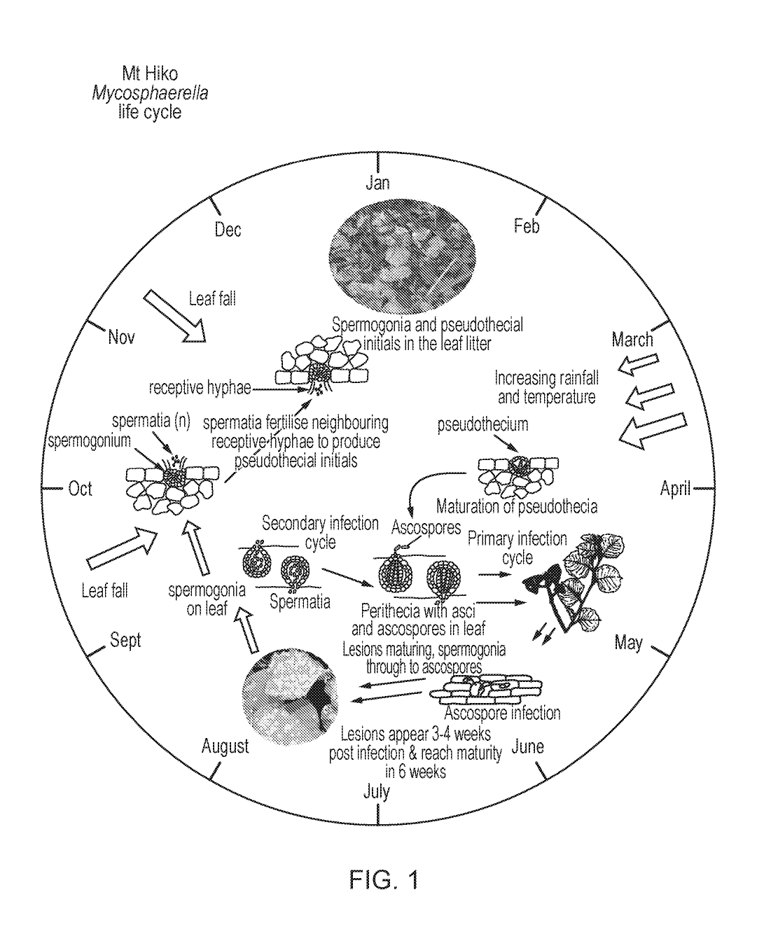

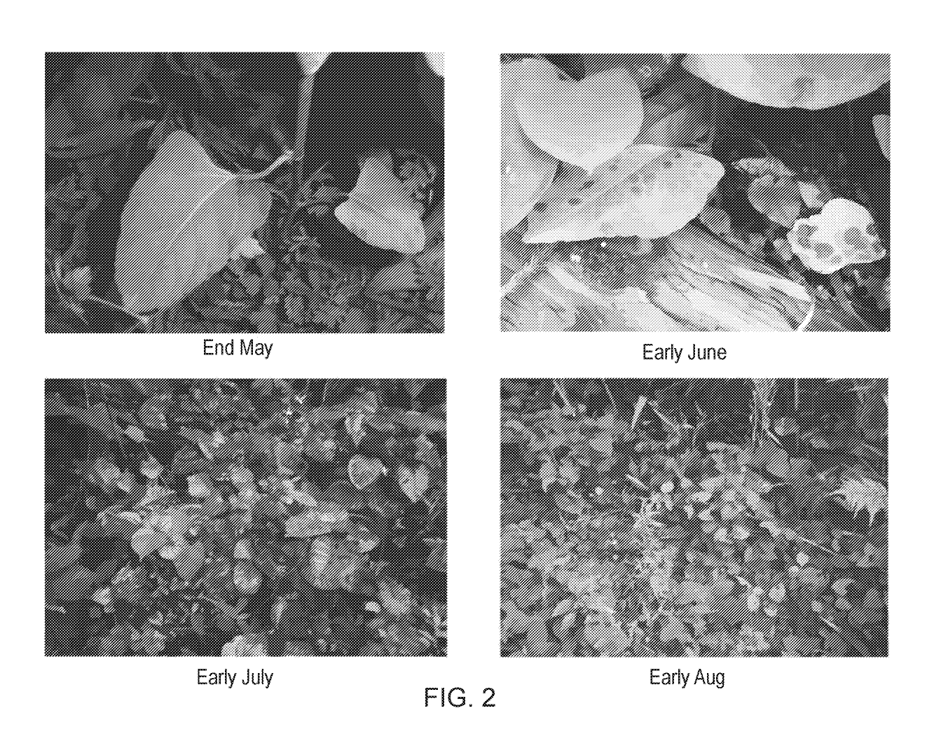

The ascomycete Mycosphaerella polygoni-cuspidati (Mycosphaerellaceae, scomycota), or Mycosphaerella leaf-spot, is a fungal pathogen associated with Japanese knotweed (Fallopia japonica) in its native range in Japan. It has been shown that the pathogen is widespread in Japan causing extensive damage to its host and to exhibit apparent field host-specificity. FIG. 1 describes the life cycle of the fungus and FIG. 2 shows the disease symptoms of the fungus on Japanese knotweed at the end of May, in early June, in early July and in early August. The typical symptoms of the disease caused by the pathogen are chestnut brown circular lesions on the upper leaf surface which are surrounded by a darker, water-soaked halo. With progressing infection these individual lesions coalesce. On the lower leaf surface the appearance of the lesions is pale brown with a prominent grayish-brown halo and whitened veins. Heavy infection results in extensive leaf necrosis, leaf distortion and premature defoliation. Most commonly observed in the necrotic lesions are spermogonia as the first fruiting bodies appearing in the life-cycle. Spermogonia are dark brown to black, immersed to semi-erumpent and appear sometimes in concentric rings within lesions. They occur on both leaf surfaces but are more prominent on the upper leaf surface. Ascomata or pseudothecia as the fruiting bodies appearing subsequently in the life cycle, are less frequent, they are larger and more erumpent than the spermogonia.

The ascomycete life cycle generally consists of two stages: a sexual stage during which ascospores are produced as a result of spermatial mating and recombination, as well as an asexual stage characterized by the production of high numbers of conidia for dispersal. Mycosphaerella polygoni-cuspidati is known to produce exclusively the sexual ascospore stage, whilst conidia are lacking from the life cycle. Molecular studies using specifically developed markers have further proved the fungus to be heterothallic meaning that mating between two different isolates with complementary spermatial sex gametes (mating type 1 and mating type 2) needs to take place in order for infective ascospores to be produced. Greenhouse inoculation studies with Japanese knotweed using one single isolate (i.e. mating type) of the pathogen have confirmed that while infection will evoke characteristic diseases symptoms including the development of spermogonia producing spermatia, the formation of ascospores is lacking and the life cycle cannot be completed.

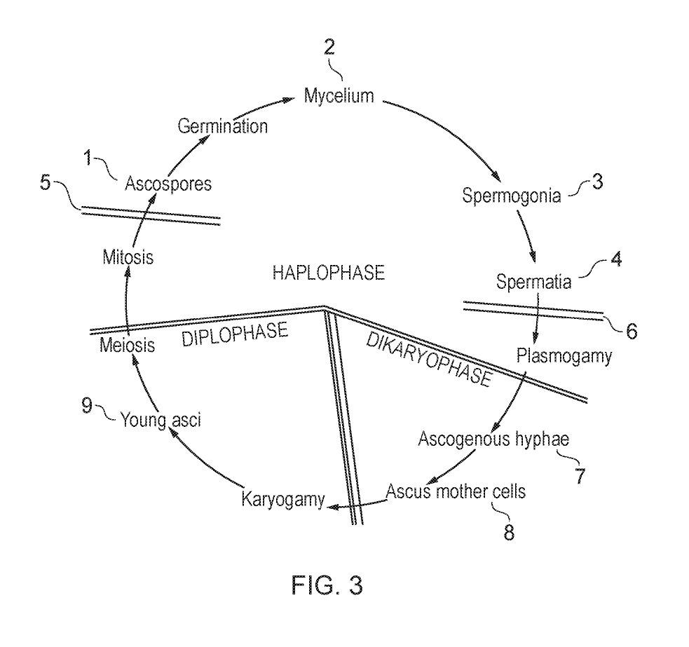

Referring to FIG. 3, the life cycle pattern of M. polygoni-cuspidati is shown. Ascospores 1 of the fungus germinate to form mycelium 2 which infects leaves of the target (typically Fallopia Japonica) entering via the stomata. As a consequence of infection, circular brown lesions develop on the leaves from necrotic spots. From the lesions, spermogonia 3 grow from which spermatia 4 are produced. M. polygoni-cuspidati is heterothallic and the two mating types of M. polygoni-cuspidati are mating type 1 and mating type 2. Spermatia of mating type 1 and spermatia of mating type 2 contact with respective, receptive hyphae 7 which are thereby fertilized to produce ascus mother cells 8. The ascus mother cells 8 produce diploid young asci 9 which, through the processes of meiosis and mitosis, form the ascospores 1 at which point the cycle repeats.

It is to be noted that M. polygoni-cuspidati has no asexual stage to its life cycle. In particular, M. polygoni-cuspidati does not form conidia, which are the asexual spores of most ascomycetes. Conidia as the asexual stage of an Ascomycete have never been observed in the field, following greenhouse inoculation or in culture.

In embodiments of the present invention, cells of M. polygoni-cuspidati are provided. It is to be understood that the cells may be of any strain of M. polygoni-cuspidati. Such varieties can be characterized as having at least 98%, preferably at least 99% similiarity (identity) in the ITS region to the ITS region of M. polygoni-cuspidati as identified in Kurose et al and having the IMI number 401912 and the GenBank accession number AB 434912. Furthermore, the species is heterothallic, has no asexual stage and has the ability to infect F. japonica.

A taxonomic description of Mycosphaerella polygoni-cuspidati is provided in Kurose et al., (2009) Systematics of Mycosphaerella species associated with the invasive weed Fallopia japonica, including the potential biological control agent M. polygoni-cuspidati. Mycoscience 50, 179-189. The fungus was originally described by Hara, Byotyugai Zasshi 5: 617, 1918, but the specimen was lost, hence this pathogen was re-described with a neotype deposited under the IMI number 393529. Hara et al and Kurose et al. and, in particular the taxonomic description of M. polygoni-cuspidati therein, are hereby incorporated by reference.

As explain above, M. polygoni-cuspidati has two mating types and the unique mating type sequences of Mycosphaerella polygoni-cuspidati are shown below.

MAT1-1-1 sequence (1,296 bp)
(SEQ. ID NO: 1)
ATGGCTACCCAAGCCCTGCCAGACACCCTTGACACCCTTCGCGACCTCTT
CGCGACCGTCCCCGAGGAGATGGTTACCCAGCTCGTTTCTGAGGTCACGG
CTGCGCTGAGCGAAGCAGCAGCTCGACGAGCCACAGCACCGAGACGCACC
GCACATGGCGCAATGCCCGGCACGACTGCCACCACCTCGGCTGTAGCGAA
GACGCAGCGTGTGGCCGGTGGGCCAAAGAGACCGCTCAACTCCTGGATGG
CATTCCGCAGTATGTCCACCTCCCTCCTTGCTCTTGTCTCTTACTGACTT
CAACACAGAGTTCTACAACGGCGCGCTCTCGCCGCACACTCAGAAAGTGA
TCTCCAAAGCCCTGACGAAGTTTTGGCGTGAGGACCCCTTCGGCGCCAAG
TGGTTCGTAACTCCATGTTCTTCACCTGACACTGAAGCATCTGCTAACAT
ATCGCAGGGTCATTCTCGCCAAGGCATACAGTGTCGTGCGCGGAAGCCGC
GAGAAGCAAGAGGCTCCGCTTGACGAGTTCTTCATCATCTGCGCACCACT
CATCGGCGTCATTCCGCCAAACGAGTACATGCAGCAGATGGGATGGCATC
TCGGCCCGCCAATGGAGGGCGACAAGGTACCGTGCCCCCCGCAGCCCCTT
CGTTGCCTCAGTGCTGACCTCTACCTAGGAGAAGATGCCGCAGATCACTC
GCCTCTTCATCCCAAACCTCGACACCTTTCCTGATGCGATCACGATGACC
ACACTCAGTGTCGACGATCTGGTCCAGGCCTGCTACAACGCCGGCTACGT
CGACGCCAGCAACGACGACTTGCCGAGCGCAAGCACCCAGGGCTCACTCA
CCATGGTCGCCCGGCCAACTCCCGTCACCAACGCCGACATGGCTGTGACC
ACACATGTGACACCCGTCGCTCAGACTGACATCACCAACCCCGACGTCGA
GCCAGATCTTGAGCCAGAGTTCGAGCCAGATTTCGAGCCATTCAACGACC
ACCGCCCTGTCCCACTCGACCATGGTCAGTACGCACAACAGACCGTCACG
CCCGCTCAGCTGTCCAACAACGAGGCCATCGAGATCGTCCAGTACCACGG
CGACGAAGACCTCGGCCTGTTCTTCAACCCTGTCGACGGCATGTTCGGCA
CCTTCTTCAACCCAAACGAGAACAACGACGAATTCGCCGCCTTCGACATG
GACATCTACAACCACGCCCCGCACACTACCGCAAACGAGGTCCAGCAGTC
TGTCCAGAACGCCTCCCTCGACAACACCTTCTACTACGACGGTTGA

In a first embodiment, analysis of the cDNA MAT1-1-1 sequence indicates that its structure is as follows.

cDNA (exon): 1-259, 309-402, 458-626, 679-1,296 bp
intron: 260-308, 403-457, 627-678 bp
α-box: 223-259, 309-402, 458-623 bp

Therefore the amino acid sequence of MAT1-1-1 encoded by the nucleotide sequence of SEQ. ID NO: 1 can be predicted as follows.

(SEQ. ID NO: 2)
MATQALPDTLDTLRDLFATVPEEMVTQLVSEVTAALSEAAARRATAPRRT
AHGAMPGTTATTSAVAKTQRVAGGPKRPLNSWMAFRKFYNGALSPHTQKV
ISKALTKFWREDPFGAKWILAKAYSVVRGSREKQEAPLDEFFIICAPLIG
VIPPNEYMQQMGWHLGPPMEGDKEKMPQITRLFIPNLDTFPDAITMTTLS
VDDLVQACYNAGYVDASNDDLPSASTQGSLTMVARPTPVTNADMAVTTHV
TPVAQTDITNPDVEPDLEPEFEPDFEPFNDHRPVPLDHGQYAQQTVTPAQ
LSNNEAIEIVQYHGDEDLGLFFNPVDGMFGTFFNPNENNDEFAAFDMDIY
NHAPHTTANEVQQSVQNASLDNTFYYDG

Sequence of α-box region in MAT1-1-1 as follows.

(SEQ. ID NO: 3)
CCAAAGAGACCGCTCAACTCCTGGATGGCATTCCGCAAGTTCTACAACGG
CGCGCTCTCGCCGCACACTCAGAAAGTGATCTCCAAAGCCCTGACGAAGT
TTTGGCGTGAGGACCCCTTCGGCGCCAAGTGGGTCATTCTCGCCAAGGCA
TACAGTGTCGTGCGCGGAAGCCGCGAGAAGCAAGAGGCTCCGCTTGACGA
GTTCTTCATCATCTGCGCACCACTCATCGGCGTCATTCCGCCAAACGAGT
ACATGCAGCAGATGGGATGGCATCTCGGCCCGCCAATGGAGGGCGAC

Furthermore, the amino acid sequence of α-box in MAT1-1-1 encoded by the nucleotide sequence of SEQ. ID NO: 3 can be predicted as follows.

(SEQ. ID NO: 4)
PKRPLNSWMAFRKFYNGALSPHTQKVISKALTKFWREDPFGAKWVILAK
AYSVVRGSREKQEAPLDEFFIICAPLIGVIPPNEYMQQMGWHLGPPMEGD

In a second embodiment, analysis of the cDNA MAT1-1-1 sequence indicates that its structure is as follows.

cDNA (exon): 1-259, 309-402, 458-626, 679-1,296 bp
intron: 260-308, 403-457, 627-678 bp
α-box: 225-259, 309-402, 458-625 bp

Sequence of α-box region in MAT1-1-1 as follows.

(SEQ. ID NO: 13)
AAAGAGACCGCTCAACTCCTGGATGGCATTCCGCAAGTTCTACAACGGCG
CGCTCTCGCCGCACACTCAGAAAGTGATCTCCAAAGCCCTGACGAAGTTT
TGGCGTGAGGACCCCTTCGGCGCCAAGTGGGTCATTCTCGCCAAGGCATA
CAGTGTCGTGCGCGGAAGCCGCGAGAAGCAAGAGGCTCCGCTTGACGAGT
TCTTCATCATCTGCGCACCACTCATCGGCGTCATTCCGCCAAACGAGTAC
ATGCAGCAGATGGGATGGCATCTCGGCCCGCCAATGGAGGGCGACAA

In a third embodiment, analysis of the cDNA MAT1-1-1 sequence indicates that its structure is as follows.

cDNA (exon): 1-259, 309-402, 458-626, 679-1,296 bp
intron: 260-308, 403-457, 627-678 bp
α-box: 223-259, 309-402, 458-626, 679-800 bp

Sequence of α-box region in MAT1-1-1 as follows.

(SEQ. ID NO: 14)
CCAAAGAGACCGCTCAACTCCTGGATGGCATTCCGCAAGTTCTACAACGG
CGCGCTCTCGCCGCACACTCAGAAAGTGATCTCCAAAGCCCTGACGAAGT
TTTGGCGTGAGGACCCCTTCGGCGCCAAGTGGGTCATTCTCGCCAAGGCA
TACAGTGTCGTGCGCGGAAGCCGCGAGAAGCAAGAGGCTCCGCTTGACGA
GTTCTTCATCATCTGCGCACCACTCATCGGCGTCATTCCGCCAAACGAGT
ACATGCAGCAGATGGGATGGCATCTCGGCCCGCCAATGGAGGGCGACAAG
GAGAAGATGCCGCAGATCACTCGCCTCTTCATCCCAAACCTCGACACCTT
TCCTGATGCGATCACGATGACCACACTCAGTGTCGACGATCTGGTCCAGG
CCTGCTACAACGCCGGCTACGT

Furthermore, the amino acid sequence of α-box in MAT1-1-1 encoded by the nucleotide sequence of SEQ. ID NO: 14 can be predicted as follows (based on Conserved Domain Database, NCBI which can be accessed on the internet using the URL http://www.ncbi.nlm.nih.gov/cdd/).

(SEQ. ID NO: 15)
PKRPLNSWMAFRKFYNGALSPHTQKVISKALTKFWREDPFGAKWVILAKA
YSVVRGSREKQEAPLDEFFIICAPLIGVIPPNEYMQQMGWHLGPPMEGDK
EKMPQITRLFIPNLDTFPDAITMTTLSVDDLVQACYNAGY
MAT1-2-1 (1,087 bp)
(SEQ. ID NO: 5)
ATGACCACCAGCATCACCACCCACCCCCCGCAGGCGCCGCTGCCAGAGCT
CGACCAGTTCTTCCTGAATGAGACTCGCATCACCGACTACATCGTCGAGC
AGCGCGTCTGTGGTGCTGACATCATCCGCATCCCCCTAGACGAGGCCCGT
GGCTGTGAGAGCGCCCGTATCAAGAGTGCCGTCGAGTAAGTCCATCAGCC
GTCTTCACATGTTACGGTTCCCGACTGACCTCTCATAGGCACATGACCAA
CACCAGCTTCGAGGTCGTCGTCAGCCTCCCGACGTGGACAGTTGACTTCA
TCGCTCAAGGCCCCATGCCGCCCAGCCCGCCCTCGGTCAAGGTGCCTCGC
CCAAAGAACGCCTTTGTGATCTACCGCCTTGCCAACCACCAGCGGGTCAA
GGCAGCGAATCCTGATCTGCACAACAACGAGATCTGTAAGCATTTCTCTT
GCCCCGCCGTGTCAGACTACAACTAACACATCTCTCAGCCAAGATCATCG
GCGCCATGTGGGGTCGCGAGAGCCCAGACGTCAAAGACGACTTCAGAGAC
CAAGCTGAGGAAGAGAAGCGTCTTCACGCTCTCGCTCACCCAGGCTACCA
GTATCAGCCACGCAAGCCTTCTGAGAAGAAGAAGCGCATGACCAAGAACA
AGCTGGCCAAGATCGCAGCGAGAGCTCGCAGCGCCGGCGATGACATGGAT
GCGACCACCCCACCACCCCAGGTCGCTCACCCGCTCGTCACATTCAACGA
CGACGCTCCAGACCGTGTGCCTTTCAACTTCTCTGCTGCCCTCGCCCCAC
CGCAGTACGCCGCTCCTCAGGTCGATGCCTACGTCCCAGATGAGTATGCC
GAGCAGATTCGCCAGGCCGAGCTCGACAACACCAACTTCGACCGTGCCGA
AGCTGCCCTCGACGCTGACTTCAGCGCATTCGATGCTGCCATCCACAACG
TCGCTGGCAATGTCGACAACCCAGTCGAATCTTTCGACGCCAACAGCTTC
AACGCCGGCGACTTCTACGACCCCTCCTTCAACGTCGACACGTTCTTCGA
CTTCACCGCCGCCAGCGGCTTCGTCTACAACGGTTAA

In a first embodiment, analysis of the cDNA MAT1-2-1 sequence indicates that its structure is as follows.

cDNA (exon): 1-185, 239-435, 489-1,087 bp
intron: 186-238, 436-488 bp

### HMG box: 339-435, 489-622 bp

Therefore the amino acid sequence of MAT1-2-1 encoded by the nucleotide sequence of SEQ. ID NO: 5 can be predicted as follows.

(SEQ. ID NO: 6)
MTTSITTHPPQAPLPELDQFFLNETRITDYIVEQRVCGADIIRIPLDEAR
GCESARIKSAVEHMTNTSFEVVVSLPTWTVDFIAQGPMPPSPPSVKVPRP
KNAFVIYRLANHQRVKAANPDLHNNEISKIIGAMWGRESPDVKDDFRDQA
EEEKRLHALAHPGYQYQPRKPSEKKKRMTKNKLAKIAARARSAGDDMDAT
TPPPQVAHPLVTFNDDAPDRVPFNFSAALAPPQYAAPQVDAYVPDEYAEQ
IRQAELDNTNFDRAEAALDADFSAFDAAIHNVAGNVDNPVESFDANSFNA
GDFYDPSFNVDTFFDFTAASGFVYNG

Sequence of HMG box region in MAT1-2-1 as follows.

(SEQ. ID NO: 7)
AAGGTGCCTCGCCCAAAGAACGCCTTTGTGATCTACCGCCTTGCCAACCA
CCAGCGGGTCAAGGCAGCGAATCCTGATCTGCACAACAACGAGATCTCCA
AGATCATCGGCGCCATGTGGGGTCGCGAGAGCCCAGACGTCAAAGACGAC
TTCAGAGACCAAGCTGAGGAAGAGAAGCGTCTTCACGCTCTCGCTCACCC
AGGCTACCAGTATCAGCCACGCAAGCCTTCT

Furthermore, the amino acid sequence of HMG box in MAT1-2-1 encoded by the nucleotide sequence of SEQ. ID NO: 7 can be predicted as follows.

(SEQ. ID NO: 8)
KVPRPKNAFVIYRLANHQRVKAANPDLHNNEISKIIGAMWGRESPDVKDD
FRDQAEEEKRLHALAHPGYQYQPRKPS

In a second embodiment, analysis of the cDNA MAT1-2-1 sequence indicates that its structure is as follows.

cDNA (exon): 1-185, 239-435, 489-1,087 bp
intron: 186-238, 436-488 bp

### HMG box: 341-435, 489-624 bp

Sequence of HMG box region in MAT1-2-1 as follows.

(SEQ. ID NO: 16)
GGTGCCTCGCCCAAAGAACGCCTTTGTGATCTACCGCCTTGCCAACCACC
AGCGGGTCAAGGCAGCGAATCCTGATCTGCACAACAACGAGATCTCCAAG
ATCATCGGCGCCATGTGGGGTCGCGAGAGCCCAGACGTCAAAGACGACTT
CAGAGACCAAGCTGAGGAAGAGAAGCGTCTTCACGCTCTCGCTCACCCAG
GCTACCAGTATCAGCCACGCAAGCCTTCTGA

In a third embodiment, analysis of the cDNA MAT1-2-1 sequence indicates that its structure is as follows.

cDNA (exon): 1-185, 239-435, 489-1,087 bp
intron: 186-238, 436-488 bp

### HMG box: 348-435, 489-622 bp

Sequence of HMG box region in MAT1-2-1 as follows.

(SEQ. ID NO: 17)
CGCCCAAAGAACGCCTTTGTGATCTACCGCCTTGCCAACCACCAGCGGGT
CAAGGCAGCGAATCCTGATCTGCACAACAACGAGATCTCCAAGATCATCG
GCGCCATGTGGGGTCGCGAGAGCCCAGACGTCAAAGACGACTTCAGAGAC
CAAGCTGAGGAAGAGAAGCGTCTTCACGCTCTCGCTCACCCAGGCTACCA
GTATCAGCCACGCAAGCCTTCT

Furthermore, the amino acid sequence of α-box in MAT1-1-1 encoded by the nucleotide sequence of SEQ. ID NO: 17 can be predicted as follows (based on Conserved Domain Database, NCBI which can be accessed on the internet using the URL http://www.ncbi.nlm.nih.gov/cdd/).

(SEQ. ID NO: 18)
RPKNAFVIYRLANHQRVKAANPDLHNNEISKIIGAMWGRESPDVKDDFR
DQAEEEKRLHALAHPGYQYQPRKPS

Risk of Crossing/Hybridization Between M. polygoni-Cuspidati and Other Fungal Pathogens

It is considered unlikely that M. polygoni-cuspidati could cross or hybridize with other fungal pathogens or Mycosphaerella species in the UK. The taxonomic name and position of the pathogen are as follows: Mycosphaerella polygoni-cuspidati (Mycosphaerellaceae, Mycosphaerellales, Dothideomycetidae, Dothideomycetes, Ascomycota, Fungi). Morphological characteristics place the leaf-spot pathogen firmly in the genus Mycosphaerella. Based on morphological data, specifically ascus and ascospore dimensions, as well as molecular data, this leaf spot also falls within the species concept, as described by Hara (1918) from leaf litter from the island of Honshu. But, as no type was designated a neotype description has been published (Kurose et al., 2009) Molecular analysis showed M. polygoni-cuspidati sequences (ITS region) to have no close match to those documented in molecular databases (i.e. GenBank). Mycosphaerella sumatrensis, a recently described species on Eucalyptus from Indonesia, showed the nearest match (96-97% homology in ITS region), thus indicating it to be a different species. The low level of homology between M. polygoni-cuspidati and M. sumatrensis might indicate that the species on Fallopia japonica var. japonica is taxonomically isolated. A BLAST search gave matches with a number of other Mycosphaerella species and their anamorphs but none of these showed more than 90% homology (i.e. Mycosphaerella areola 89%, Mycosphaerella microsora 89%, Mycosphaerella brassicicola 88%).

In the field, fungal species are known to hybridize with closely related species. “The key question, whether a newly formed fungal hybrid will persist in nature, is directly related to the relative fitness of the hybrid” (Schardl & Craven, 2003, Interspecific hybridization in plant-associated fungi and oomycetes: a review. Molecular Ecology 12: 2861-2873). Hybridization resulting in more virulent hybrids has been shown to occur in the genera Phytophthora, Ophiostoma and Melampsora (Brasier, 2001, Rapid evolution of introduced plant pathogens via interspecific hybridization. BioScience 51: 123-133; Schardl & Craven, 2003). On the other hand, based on research undertaken with Heterobasidion annosum, Garbelotto et al. (2007) Ecological constraints limit the fitness of fungal hybrids in the Heterobasidion annosum species complex. Applied Environmental Microbiology, 73: 6106-6111 propose that “in fungal species complexes characterized by differential host specificity, hybrids may not be generally unfit but may be at an ecological disadvantage with regard to their parents in a habitat specific for one of the parents.”

Thus molecular data strongly indicate that there are no closely-related Mycosphaerella species with which to hybridise. Furthermore, by using a single mating type isolate for application as a mycoherbicide M. polygoni-cuspidati will not be able to produce ascospores in the field, thus will neither reproduce nor spread beyond the area of application. Given that no other Mycosphaerella species is known to attack Japanese knotweed outside its native range Japan and M. polygoni-cuspidati will only reside in treated Japanese knotweed plants, hybridization will not take place.

Genus Fallopia

The genus Fallopia comprises around a dozen species of flowering plants in the family Polygonaceae. In embodiments of the present invention, invasive plants of the genus are preferred and plants of the species Fallopia japonica (which is commonly known as “Japanese knotweed”) are particularly preferred. Plants of the variety Fallopia japonica var. japonica are more particularly preferred. However, other embodiments of the invention relate to plants of the species Fallopia sachalinensis (which is commonly known as “giant knotweed”). Still further embodiments relate to plants of the hybrid Fallopia×bohemica which is a hybrid of the species Fallopia japonica and Fallopia sachalinensis. It is to be understood that the invention also relates to other species within the genus Fallopia such as hybrids of Fallopia japonica with species other than Fallopia sachalinensis.

Herbicidal Product

The principal component of the herbicidal product of the invention is a consortium of cells. In one embodiment of the present invention, the herbicidal products comprises a consortium of cells of M. polygoni-cuspidati or the like. In some embodiments, the consortium comprises only cells of a single species but in other embodiments, the consortium comprises cells of a plurality of species such as, for example, i) cells of a first strain of M. polygoni-cuspidati, ii) cells of a second strain of M. polygoni-cuspidati and iii) cells of a different species. It is to be understood that all of the cells of M. polygoni-cuspidati and any other cells which are capable of mating with M. polygoni-cuspidati are of the same mating type (i.e. either all are mating type 1 or all are mating type 2). However, other cells, which do not mate with M. polygoni-cuspidati, may, if they have a mating type, be of any mating type.

It is preferred that the cells in the consortium are mycelium cells. It is to be understood that these are the haploid mycelial cells that arise from ascospores' germination from and not mycelia from the dikaryophase of the life cycle. In one embodiment, the cells in the consortium are individual, separate cells but in other embodiments, the cells are in the form of part or all of a mycelium or mycelia. In further embodiments, the cells of the consortium are ascospores and in still further embodiments the cells are a mixture of mycelium cells and ascospores.

In one embodiment, the consortium of cells is provided as follows. Infected plants are readily available in Japanese knotweed stands at Mount Hiko. Collections of the pathogen are made by sampling infected leaves displaying the above described disease symptoms. Leaves are dried in a plant press for 3-5 days with a daily change of newspaper as the drying paper and then transferred into wax packets.

Using a stereomicroscope, the leaf spots are assessed for mature pseudothecia of M. polygoni-cuspidati. Leaf discs containing these pseudothecia are cut using a hole-punch and immersed in sterile distilled water at 19° C. for 24 hours. Leaf discs (pseudothecia facing down) are affixed to the lid of a Petri dish using double sided sticky tape, suspended over tap water agar (TWA) and incubated at 19° C. for 24 hours. After this incubation period plates are microscopically examined for ascospore discharge. Mycosphaerella polygoni-cuspidati ascopores are identified based on morphology and single spore isolates are made by removing individual ascospores together with a block of agar using a sterile hypodermic needle. Each block is placed onto separate TWA agar plates and placed in an incubator at 19° C. Each single spore isolate is then molecularly characterized with respect to its mating type.

Multiplex PCR is performed on samples of each spore isolate. Primer sets for differentiating the two mating types are shown below. These primers have been designed originally based on the sequence of each mating type shown above.

Primer sets for detecting Mating type 1-1 isolates

Forward primer:
(SEQ. ID NO: 9)
GTTACCCAGCTCGTTTCTGA
Reverse primer:
(SEQ. ID NO: 10)
AGTGATCTGCGGCATCTTCT

Primer sets for detecting Mating type 1-2 isolates

Forward:
(SEQ. ID NO: 11)
CGCCTTTGTGATCTACCGCC
Reverse:
(SEQ. ID NO: 12)
GCCAGCTTGTTCTTGGTCAT

However, it is to be appreciated that mating type characterization may be performed in accordance with alternative methods, for example by detecting the mating type sequences using different sets of primer sequences that are specific for each mating type isolate.

After mating type characterization, a mycelial culture is grown up which contains the consortium of cells of the invention. In some embodiments, multiple mycelia cultures of the same mating type are mixed together in order to provide sufficient quantities of cells.

In some embodiments, the herbicidal product also comprises such further additives and/or adjuvants, in addition to the consortium of cells, as are appropriate to provide a practical crop protection product. Additives are provided to maintain the long-term viability of the consortium's propagule and to facilitate ease of handling, storage and application. In preferred embodiments, additives are provided so that the herbicidal product is in the form of a liquid or solution. For example, in one embodiment, the mycelium grown in potato dextrose broth and thus the broth itself may be used in order that the herbicidal product is in the form of a liquid. In another embodiment, the consortium comprises an additive that enhances adherence of the consortium to plant leaves. In another embodiment, the consortium comprises an additive, such as a wetting agent, that maintains the cells in an aqueous environment for an extended period such as at least 12 hours and preferably at least 24 hours. Adjuvants are provided to improve spray delivery of the consortium or enhance the pathological impacts of the propagules. Further details of suitable additives and adjuvants are provided in Fungi As Biocontrol Agents: Progress Problems and Potential edited by T. M. Butt, C. Jackson, N. Magan, which is incorporated herein by reference.

In some embodiments the concentration is 2×10⁵ mycelial fragments per ml. However, in other embodiments, the concentration is between 1×10⁴ and 1×10⁶ mycelial fragments per ml, preferably between 1×10⁵ and 3×10⁵ mycelial fragments per ml

Use of the Consortium

In use, the consortium is inoculated on the leaves of a plant of the genus Fallopia as will now be described. In some embodiments, the herbicidal product comprising the consortium is painted on to the leaves of the plant using a brush. This provides very effective coverage of the leaves but is time consuming and is not appropriate for large scale coverage. Accordingly, in preferred embodiments, the herbicidal product is sprayed on to the leaves of the plant, which permits coverage of multiple plants substantially simultaneously.

The cells of the consortium then infect the plant via the leaf stomata and there is fungal growth within the plant. Subsequently, the fungal infection results in defoliation of the plant resulting in death of the plant or a severe setback to its growth which allows other plant species the opportunity to out-compete it during the growing season. Referring to FIG. 3, it is to be appreciated that the life cycle of the fungus starts (in the production phase) at the initiation line 5 with the provision of ascospores 1. Furthermore, since only one cell mating type is present in the consortium, the life cycle does not proceed beyond the formation of spermatia 4 and thus stops at the termination line 6. This is very significant since, unlike spores, mycelia cannot be spread by wind and so plant infection is limited to contact only (i.e. the plant initially infected and any other plant where there is leaf-to-leaf contact). As such the effective area of the fungus is controlled and spread is prevented.

Since a single application of the herbicidal product may not result in plant death, in some embodiments of the invention, multiple applications of the herbicidal product are inoculated on the plant. Symptoms on the plant leaf are normally visible after 3-4 weeks and it is preferred that such symptoms are assessed before a second application of the product. Therefore, in some embodiments further applications are applied between 1 and 2 months are the preceding application. The number of applications required depends upon various factors such as the age of the plant and the climate.

While it is generally desirable to ensure that spraying covers only leaves of plants of the genus Fallopia, herbicidal product that does fall on other plants is not generally of concern since very few other plants are susceptible to infection by the fungus of the present invention. Furthermore, even if a non-target plant were infected by the fungus of the invention, it would at worst result in infection of plants in physical contact with the originally-infected plant since the presence of only a single mating type in the consortium means that the fungus cannot complete its life cycle to form ascospores. Indeed, spread of the fungus is limited in most circumstances to growth within the plant originally inoculated.

### EXAMPLES

### Example 1

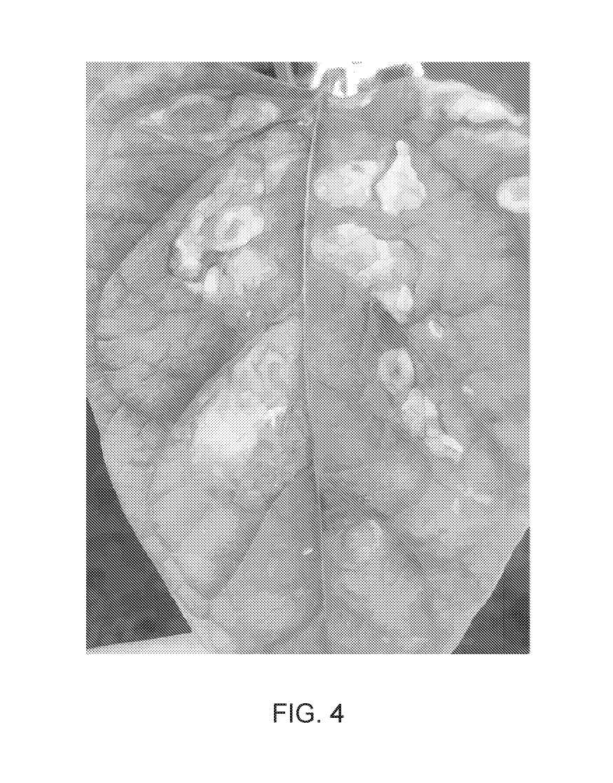

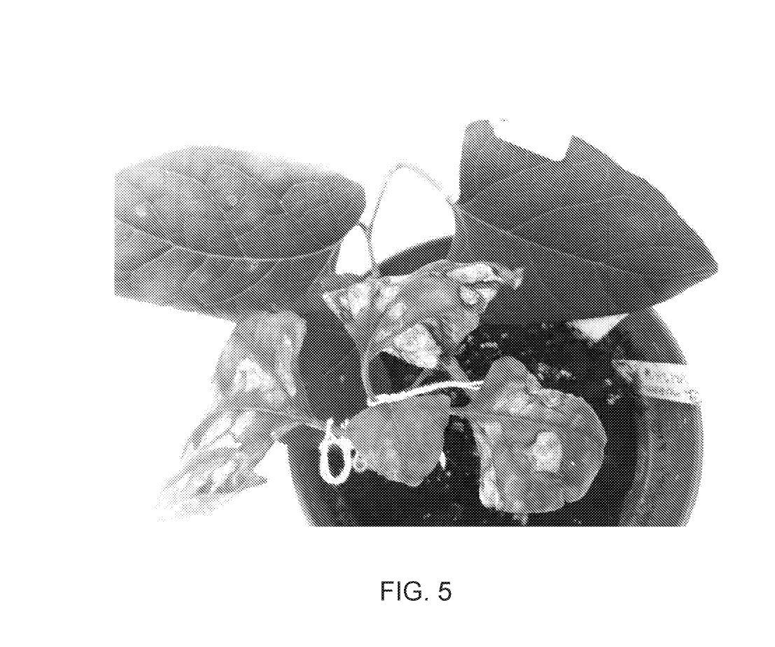

During survey work in Japan M. polygoni-cuspidati was commonly encountered in Japanese knotweed stands at Mount Hiko. Samples of M. polygoni-cuspidati were taken and ascospores thereof were inoculated onto leaves of Japanese knotweed in a quarantine greenhouse. FIG. 4 shows a leaf 2.5 months after inoculation. FIG. 5 shows a plant 2.5 months after inoculation in which the top leaf with a white label over it is the control leaf.

### Example 2

During survey work in Japan M. polygoni-cuspidati was commonly encountered in Japanese knotweed stands at Mount Hiko. Collections of the pathogen were made by sampling infected leaves displaying the above described disease symptoms. Leaves were dried in a plant press for 3-5 days with a daily change of newspaper as the drying paper and then transferred into wax packets. Samples were taken back into quarantine in the UK under licences.

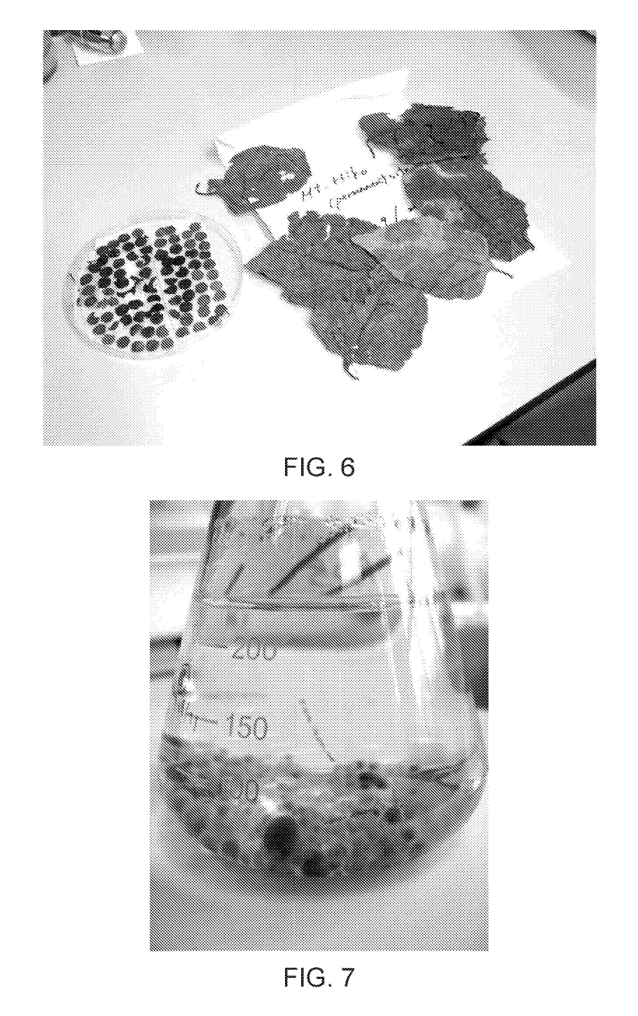

Using a stereomicroscope, the leaf spots were assessed for mature pseudothecia of M. polygoni-cuspidati. Leaf discs containing these pseudothecia were cut using a hole-punch and immersed in sterile distilled water at 19° C. for 24 hours. Leaf discs (pseudothecia facing down) were affixed to the lid of a Petri dish using double sided sticky tape (see FIG. 6), suspended over tap water agar (TWA) and incubated at 19° C. for 24 hours. After this incubation period plates were microscopically examined for ascospore discharge. Mycosphaerella polygoni-cuspidati ascopores were identified based on morphology and single spore isolates were made by removing individual ascospores together with a block of agar using a sterile hypodermic needle. Each block was placed onto separate TWA agar plates and placed in an incubator at 19° C. Each single spore isolate can then be molecularly characterized with respect to its mating type.

### Example 3

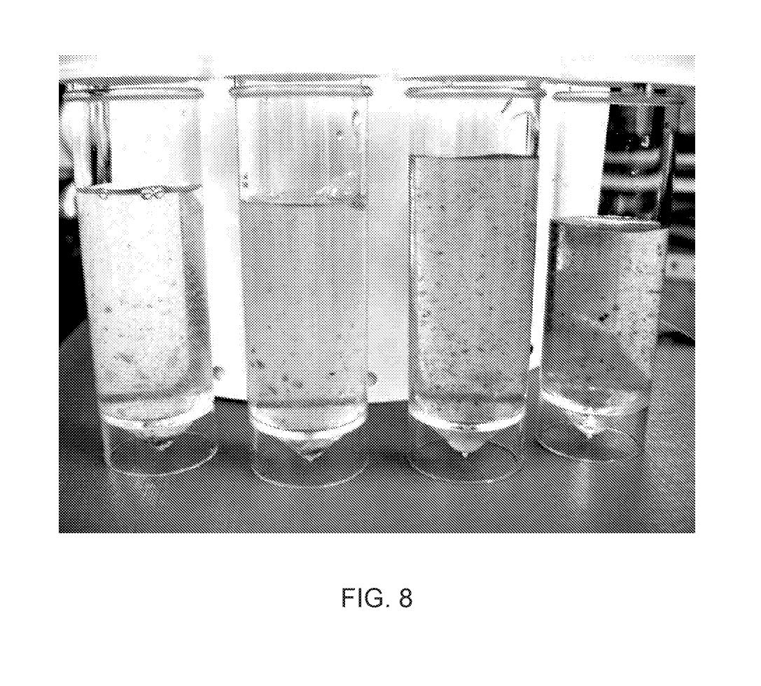

Four mycelial plugs, 5 mm in diameter, were taken from the actively growing margin of an individual culture of M. polygoni-cuspidati using a sterile cork-borer. The mycelial plugs were split into smaller fragments and added to 75 ml of potato dextrose broth (PDB) in a 250 ml conical flask. A flask containing PDB only was used as a control to detect potential contamination during the mass production process. Flasks were sealed with a sterile foam bung and placed on a rotary shaker at 170 rpm at 18° C. for 8 days (see FIG. 7). Subsequently the mycelial broth was transferred to a sterilised blender and macerated for 20 seconds in order to produce a more homogenous inoculum (FIG. 8).

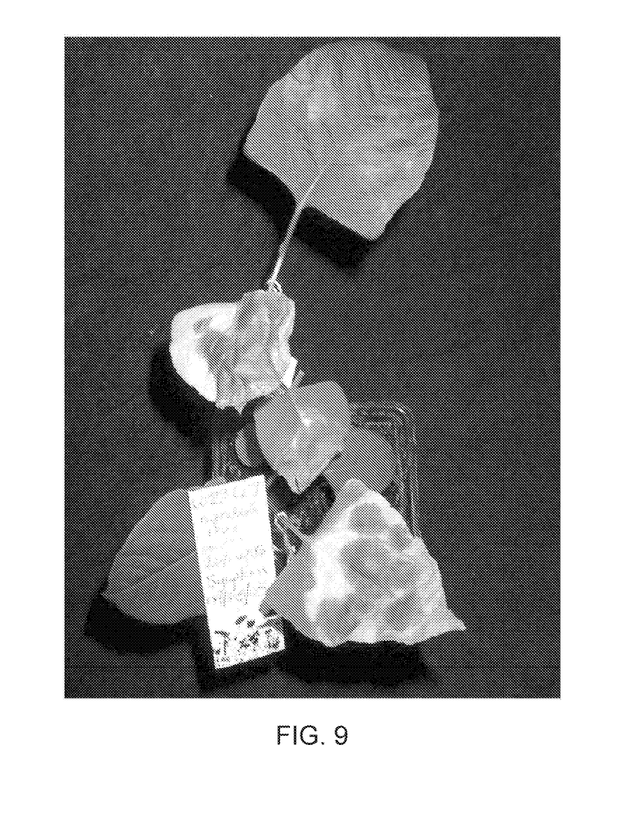

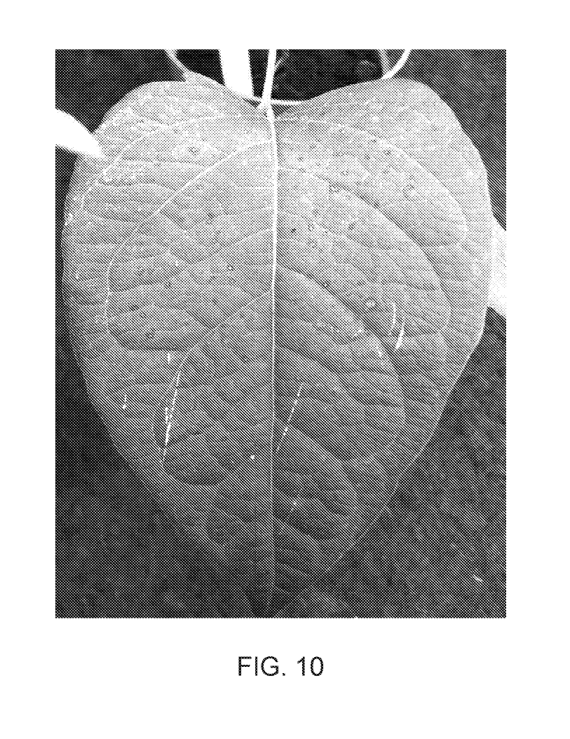

The fragmented mycelium broth was applied to plants by using a paintbrush and brushing the inoculum onto the upper and lower leaf surface. Control plants/leaves were inoculated with PDB liquid medium only. Inoculated plants were placed in a customized dew chamber (Mercia Scientific, Birmingham, UK) set at 20° C., without light for 48 h. The viability of the inoculum was assessed as growth of mycelial fragments on TWA. Upon removal from the dew chamber all plants were maintained in a designated quarantine greenhouse chamber under a controlled temperature and light regime of 20° C. and 12-h dark/12-h light and regularly monitored for the development of disease symptoms. FIG. 9 is a photograph of a leaf c. 3 weeks after inoculation with mycelial broth. FIG. 10 is a photograph of a leaf 1 month after inoculation with mycelial broth.

1-21. (canceled)

22. A method for herbicidal control of a plant of the genus Fallopia comprising contacting the plant with a composition comprising a consortium of cells of the species Mycosphaerella polygoni-cuspidati or the like, wherein the cells in the consortium are all of the same mating type.

23. The method according to claim 22, further comprising the step of maintaining the cells in an aqueous environment on the plant for at least 12 hours.

24. The method according to claim 23, wherein the cells are maintained in the aqueous environment on the plant for at least 24 hours.

25. A method according to claim 22, comprising contacting the leaves of the plant with the composition.

26. The method according to claim 22, wherein the plant is of the species or hybrid selected from the group consisting of: Fallopia japonica, Fallopia sachalinensis and Fallopia×bohemica.

27. The method according to claim 26, wherein the plant is of the variety Fallopia japonica var. japonica.

28. The method according to claim 22, wherein the cells are mycelium cells.

29. The method according to claim 22, wherein the Mycosphaerella polygoni-cuspidati cells in the consortium are all of mating type 1.

30. The method according to claim 22, wherein the Mycosphaerella polygoni-cuspidati cells in the consortium are all of mating type 2.

31. A method of producing a consortium of cells of the species Mycosphaerella polygoni-cuspidati wherein the cells in the consortium are all of the same mating type comprising the steps of:
i. isolating a first individual ascospore from a sample of ascospores of the species Mycosphaerella polygoni-cuspidati; and
ii. generating at least a first mycelium from said first isolated ascospore.

32. A method according to claim 31, wherein step ii further comprises determining the mating type of said first mycelium.

33. A method according to claim 32, wherein determining the mating type of said first mycelium comprises detecting the presence of a biological marker specific for one mating type.

34. A method according to claim 31, further comprising the steps of:
iii. isolating a second individual ascospore from a sample of ascospores of the species Mycosphaerella polygoni-cuspidati;
iv. generating at least a second mycelium from said second isolated ascospore;
v. determining the mating type of said second mycelium;
vi. confirming that the mating type of the first mycelium is the same as the mating type of the second mycelium;
vii. mixing the first and second mycelia.

35. A method according to claim 34, wherein step v comprises detecting the presence of a biological marker specific for one mating type.

36. A composition for the herbicidal control of a plant of the genus Fallopia comprising a consortium of cells of the species Mycosphaerella polygoni-cuspidati or the like, wherein the cells in the consortium are all of the same mating type.

37. A composition according to claim 36, wherein the cells are mycelium cells.

38. A composition according to claim 36, wherein the Mycosphaerella polygoni-cuspidati cells in the consortium are all of mating type 1.

39. A composition according to claim 36, wherein the Mycosphaerella polygoni-cuspidati cells in the consortium are all of mating type 2.

40. A composition according to claim 36, further comprising an adhesive agent and/or a wetting agent.


SEQUENCE LISTING (18 sequences)

SEQ ID NO: 1 - DNA, 1296 bp, Mycosphaerella polygoni-cuspidati
  CDS (1)..(259)
  CDS (309)..(402)
  CDS (458)..(626)
  CDS (679)..(1293)
atggctacccaagccctgccagacacccttgacacccttcgcgacctcttcgcgaccgtccccgaggagatggttacccagctcgtttctgaggtcacggctgcgctgagcgaagcagcagctcgacgagccacagcaccgagacgcaccgcacatggcgcaatgcccggcacgactgccaccacctcggctgtagcgaagacgcagcgtgtggccggtgggccaaagagaccgctcaactcctggatggcattccgcagtatgtccacctccctccttgctcttgtctcttactgacttcaacacagagttctacaacggcgcgctctcgccgcacactcagaaagtgatctccaaagccctgacgaagttttggcgtgaggaccccttcggcgccaagtggttcgtaactccatgttcttcacctgacactgaagcatctgctaacatatcgcagggtcattctcgccaaggcatacagtgtcgtgcgcggaagccgcgagaagcaagaggctccgcttgacgagttcttcatcatctgcgcaccactcatcggcgtcattccgccaaacgagtacatgcagcagatgggatggcatctcggcccgccaatggagggcgacaaggtaccgtgccccccgcagccccttcgttgcctcagtgctgacctctacctaggagaagatgccgcagatcactcgcctcttcatcccaaacctcgacacctttcctgatgcgatcacgatgaccacactcagtgtcgacgatctggtccaggcctgctacaacgccggctacgtcgacgccagcaacgacgacttgccgagcgcaagcacccagggctcactcaccatggtcgcccggccaactcccgtcaccaacgccgacatggctgtgaccacacatgtgacacccgtcgctcagactgacatcaccaaccccgacgtcgagccagatcttgagccagagttcgagccagatttcgagccattcaacgaccaccgccctgtcccactcgaccatggtcagtacgcacaacagaccgtcacgcccgctcagctgtccaacaacgaggccatcgagatcgtccagtaccacggcgacgaagacctcggcctgttcttcaaccctgtcgacggcatgttcggcaccttcttcaacccaaacgagaacaacgacgaattcgccgccttcgacatggacatctacaaccacgccccgcacactaccgcaaacgaggtccagcagtctgtccagaacgcctccctcgacaacaccttctactacgacggttga

SEQ ID NO: 2 - protein, 379 aa, Mycosphaerella polygoni-cuspidati
MATQALPDTLDTLRDLFATVPEEMVTQLVSEVTAALSEAAARRATAPRRTAHGAMPGTTATTSAVAKTQRVAGGPKRPLNSWMAFRKFYNGALSPHTQKVISKALTKFWREDPFGAKWVILAKAYSVVRGSREKQEAPLDEFFIICAPLIGVIPPNEYMQQMGWHLGPPMEGDKEKMPQITRLFIPNLDTFPDAITMTTLSVDDLVQACYNAGYVDASNDDLPSASTQGSLTMVARPTPVTNADMAVTTHVTPVAQTDITNPDVEPDLEPEFEPDFEPFNDHRPVPLDHGQYAQQTVTPAQLSNNEAIEIVQYHGDEDLGLFFNPVDGMFGTFFNPNENNDEFAAFDMDIYNHAPHTTANEVQQSVQNASLDNTFYYDG

SEQ ID NO: 3 - DNA, 297 bp, Mycosphaerella polygoni-cuspidati
  CDS (1)..(297)
ccaaagagaccgctcaactcctggatggcattccgcaagttctacaacggcgcgctctcgccgcacactcagaaagtgatctccaaagccctgacgaagttttggcgtgaggaccccttcggcgccaagtgggtcattctcgccaaggcatacagtgtcgtgcgcggaagccgcgagaagcaagaggctccgcttgacgagttcttcatcatctgcgcaccactcatcggcgtcattccgccaaacgagtacatgcagcagatgggatggcatctcggcccgccaatggagggcgac

SEQ ID NO: 4 - protein, 99 aa, Mycosphaerella polygoni-cuspidati
PKRPLNSWMAFRKFYNGALSPHTQKVISKALTKFWREDPFGAKWVILAKAYSVVRGSREKQEAPLDEFFIICAPLIGVIPPNEYMQQMGWHLGPPMEGD

SEQ ID NO: 5 - DNA, 1087 bp, Mycosphaerella polygoni-cuspidati
  CDS (1)..(185)
  CDS (239)..(435)
  CDS (489)..(1084)
atgaccaccagcatcaccacccaccccccgcaggcgccgctgccagagctcgaccagttcttcctgaatgagactcgcatcaccgactacatcgtcgagcagcgcgtctgtggtgctgacatcatccgcatccccctagacgaggcccgtggctgtgagagcgcccgtatcaagagtgccgtcgagtaagtccatcagccgtcttcacatgttacggttcccgactgacctctcataggcacatgaccaacaccagcttcgaggtcgtcgtcagcctcccgacgtggacagttgacttcatcgctcaaggccccatgccgcccagcccgccctcggtcaaggtgcctcgcccaaagaacgcctttgtgatctaccgccttgccaaccaccagcgggtcaaggcagcgaatcctgatctgcacaacaacgagatctgtaagcatttctcttgccccgccgtgtcagactacaactaacacatctctcagccaagatcatcggcgccatgtggggtcgcgagagcccagacgtcaaagacgacttcagagaccaagctgaggaagagaagcgtcttcacgctctcgctcacccaggctaccagtatcagccacgcaagccttctgagaagaagaagcgcatgaccaagaacaagctggccaagatcgcagcgagagctcgcagcgccggcgatgacatggatgcgaccaccccaccaccccaggtcgctcacccgctcgtcacattcaacgacgacgctccagaccgtgtgcctttcaacttctctgctgccctcgccccaccgcagtacgccgctcctcaggtcgatgcctacgtcccagatgagtatgccgagcagattcgccaggccgagctcgacaacaccaacttcgaccgtgccgaagctgccctcgacgctgacttcagcgcattcgatgctgccatccacaacgtcgctggcaatgtcgacaacccagtcgaatctttcgacgccaacagcttcaacgccggcgacttctacgacccctccttcaacgtcgacacgttcttcgacttcaccgccgccagcggcttcgtctacaacggttaa

SEQ ID NO: 6 - protein, 326 aa, Mycosphaerella polygoni-cuspidati
MTTSITTHPPQAPLPELDQFFLNETRITDYIVEQRVCGADIIRIPLDEARGCESARIKSAVEHMTNTSFEVVVSLPTWTVDFIAQGPMPPSPPSVKVPRPKNAFVIYRLANHQRVKAANPDLHNNEISKIIGAMWGRESPDVKDDFRDQAEEEKRLHALAHPGYQYQPRKPSEKKKRMTKNKLAKIAARARSAGDDMDATTPPPQVAHPLVTFNDDAPDRVPFNFSAALAPPQYAAPQVDAYVPDEYAEQIRQAELDNTNFDRAEAALDADFSAFDAAIHNVAGNVDNPVESFDANSFNAGDFYDPSFNVDTFFDFTAASGFVYNG

SEQ ID NO: 7 - DNA, 231 bp, Mycosphaerella polygoni-cuspidati
  CDS (1)..(231)
aaggtgcctcgcccaaagaacgcctttgtgatctaccgccttgccaaccaccagcgggtcaaggcagcgaatcctgatctgcacaacaacgagatctccaagatcatcggcgccatgtggggtcgcgagagcccagacgtcaaagacgacttcagagaccaagctgaggaagagaagcgtcttcacgctctcgctcacccaggctaccagtatcagccacgcaagccttct

SEQ ID NO: 8 - protein, 77 aa, Mycosphaerella polygoni-cuspidati
KVPRPKNAFVIYRLANHQRVKAANPDLHNNEISKIIGAMWGRESPDVKDDFRDQAEEEKRLHALAHPGYQYQPRKPS

SEQ ID NO: 9 - DNA, 20 bp, Artificial Sequence
  Mating type 1-1 forward primer
gttacccagctcgtttctga

SEQ ID NO: 10 - DNA, 20 bp, Artificial Sequence
  Mating type 1-1 reverse primer
agtgatctgcggcatcttct

SEQ ID NO: 11 - DNA, 20 bp, Artificial Sequence
  Mating type 1-2 forward primer
cgcctttgtgatctaccgcc

SEQ ID NO: 12 - DNA, 20 bp, Artificial Sequence
  Mating type 1-2 reverse primer
gccagcttgttcttggtcat

SEQ ID NO: 13 - DNA, 297 bp, Mycosphaerella polygoni-cuspidati
aaagagaccgctcaactcctggatggcattccgcaagttctacaacggcgcgctctcgccgcacactcagaaagtgatctccaaagccctgacgaagttttggcgtgaggaccccttcggcgccaagtgggtcattctcgccaaggcatacagtgtcgtgcgcggaagccgcgagaagcaagaggctccgcttgacgagttcttcatcatctgcgcaccactcatcggcgtcattccgccaaacgagtacatgcagcagatgggatggcatctcggcccgccaatggagggcgacaa

SEQ ID NO: 14 - DNA, 422 bp, Mycosphaerella polygoni-cuspidati
  CDS (1)..(420)
ccaaagagaccgctcaactcctggatggcattccgcaagttctacaacggcgcgctctcgccgcacactcagaaagtgatctccaaagccctgacgaagttttggcgtgaggaccccttcggcgccaagtgggtcattctcgccaaggcatacagtgtcgtgcgcggaagccgcgagaagcaagaggctccgcttgacgagttcttcatcatctgcgcaccactcatcggcgtcattccgccaaacgagtacatgcagcagatgggatggcatctcggcccgccaatggagggcgacaaggagaagatgccgcagatcactcgcctcttcatcccaaacctcgacacctttcctgatgcgatcacgatgaccacactcagtgtcgacgatctggtccaggcctgctacaacgccggctacgt

SEQ ID NO: 15 - protein, 140 aa, Mycosphaerella polygoni-cuspidati
PKRPLNSWMAFRKFYNGALSPHTQKVISKALTKFWREDPFGAKWVILAKAYSVVRGSREKQEAPLDEFFIICAPLIGVIPPNEYMQQMGWHLGPPMEGDKEKMPQITRLFIPNLDTFPDAITMTTLSVDDLVQACYNAGY

SEQ ID NO: 16 - DNA, 231 bp, Mycosphaerella polygoni-cuspidati
ggtgcctcgcccaaagaacgcctttgtgatctaccgccttgccaaccaccagcgggtcaaggcagcgaatcctgatctgcacaacaacgagatctccaagatcatcggcgccatgtggggtcgcgagagcccagacgtcaaagacgacttcagagaccaagctgaggaagagaagcgtcttcacgctctcgctcacccaggctaccagtatcagccacgcaagccttctga

SEQ ID NO: 17 - DNA, 222 bp, Mycosphaerella polygoni-cuspidati
  CDS (1)..(222)
cgcccaaagaacgcctttgtgatctaccgccttgccaaccaccagcgggtcaaggcagcgaatcctgatctgcacaacaacgagatctccaagatcatcggcgccatgtggggtcgcgagagcccagacgtcaaagacgacttcagagaccaagctgaggaagagaagcgtcttcacgctctcgctcacccaggctaccagtatcagccacgcaagccttct

SEQ ID NO: 18 - protein, 74 aa, Mycosphaerella polygoni-cuspidati
RPKNAFVIYRLANHQRVKAANPDLHNNEISKIIGAMWGRESPDVKDDFRDQAEEEKRLHALAHPGYQYQPRKPS